(19) United States
(12) Patent Application Publication
(10) Pub. No.: US 2012/0165616 A1
(43) Pub. Date: Jun. 28, 2012

(54) PORTABLE MONITORING UNIT AND A METHOD FOR MONITORING A MONITORED PERSON

(76) Inventors: Nir GEVA, Nes Ziona (IL); Yacov GEVA, London (GB)

(21) Appl. No.: 12/978,666

(22) Filed: Dec. 27, 2010

(51) Int. Cl. A61B 5/00

(52) U.S. Cl. 600/300

(57) ABSTRACT

A method and portable monitoring unit, that portable monitoring unit includes a memory module for storing a media clip that comprises instructions related to the operation of the monitoring unit, for storing raw medical information, and for storing medical event information; a processor arranged to receive raw medical information from at least one primary sensor and to process the raw medical information and detect potential medical events, to trigger a display of the media clip and to perform a potential event related operation in response to a detection of the potential medical event.

### FIELD OF THE INVENTION

This application relates to medical devices and especially to portable monitoring devices.

### BACKGROUND OF THE INVENTION

Medical events such as but not limited to high blood pressure, cardiac arrest, should be detected and taken care off as soon as possible.

There is a growing need to monitor persons in an efficient manner.

### SUMMARY OF THE INVENTION

According to an embodiment of the invention a portable monitoring unit is provided. The portable monitoring unit may include a memory module for storing a media clip that may include instructions related to the operation of the monitoring unit, for storing raw medical information, and for storing medical event information; a processor arranged to receive raw medical information from at least one primary sensor and to process the raw medical information and detect potential medical events, to trigger a display of the media clip and to perform a potential event related operation in response to a detection of the potential medical event.

The processor may be arranged to request additional information from at least one secondary sensor in response to a detection of the potential event, and to process the additional information to determine a validity of the potential medical event. The at least one primary sensor and the at least one secondary sensor may form a group of sensors.

The group of sensors may include a physiological sensor and a biomechanical sensor; and the processor may be arranged to correlate between physiological information obtained from the physiological sensor and biomechanical information obtained from the biomechanical sensor to determine a validity of the potential medical event.

The group of sensors may include an environment condition sensor and a physiological sensor; and the processor may be arranged to correlate between physiological information obtained from the physiological sensor and environment condition information obtained from the environment condition sensor to determine a validity of the potential medical event.

The group of sensors may include an environment condition sensor, a biomechanical sensor and a physiological sensor; and the processor may be arranged to correlate between physiological information obtained from the physiological sensor, biomechanical information obtained from the biomechanical sensor and environment condition information obtained from the environment condition sensor to determine a validity of the potential medical event.

The group of sensors may include two types of physiological sensors; and the processor may be arranged to correlate between physiological information obtained from the two types of physiological sensors to determine a state of a monitored person.

The portable monitoring unit may include a transmitter and the processor may be arranged to instruct the transmitter to transmit a potential event information in response to a detection of the potential medical event.

The transmitter may be arranged to attempt to transmit the potential event information and to generate a transmission failure indication of the attempt fails; the processor may be arranged to provide an alert that induces a suggestion to change a location of the monitored person or to inform a medical center of the failure by means that differ from the transmitter.

The portable monitoring unit may include a positioning module arranged to determine a position of; the transmitter may be arranged to attempt to transmit the potential event information and to generate a transmission failure indication of the attempt fails; the processor may be arranged to determine a preferred transmission location and generate an alert to a monitored person indicative of the preferred transmission location; a probability of transmission failure from the preferred transmission location may be substantially lower than a probability of a transmission failure at a current location of the monitored person.

The processor may be arranged to activate the transmitter and instruct the transmitter to perform long range transmission of the potential event information in response to a detection of the potential medical event.

The portable monitoring unit may include, may include a transmitter, a primary power source and a secondary power source; the processor may be arranged to prevent a transmission of potential event information during a low power period during which the primary power source may be empty; and to initiate a transmission of potential event information obtained during the low power period.

The processor may be arranged to receive the raw medical information in a continuous manner.

According to an embodiment of the invention a portable monitoring unit is provided. The portable monitoring unit may include a memory module for storing a media clip that may include instructions related to the operation of the monitoring unit, for storing raw medical information, and for storing medical event information; a processor arranged to receive raw medical information from at least one primary sensor and to process the raw medical information and detect potential medical events, to request additional information from at least one secondary sensor in response to a detection of the potential event, and to process the additional information to determine a validity of the potential medical event. The at least one primary sensor and the at least one secondary sensor form a group of sensors.

The group of sensors may include a physiological sensor and a biomechanical sensor; and the processor may be arranged to correlate between physiological information obtained from the physiological sensor and biomechanical information obtained from the biomechanical sensor to determine a validity of the potential medical event.

The group of sensors may include an environment condition sensor and a physiological sensor; and the processor may be arranged to correlate between physiological information obtained from the physiological sensor and environment condition information obtained from the environment condition sensor to determine a validity of the potential medical event.

The group of sensors may include an environment condition sensor, a biomechanical sensor and a physiological sensor; and the processor may be arranged to correlate between physiological information obtained from the physiological sensor, biomechanical information obtained from the biomechanical sensor and environment condition information obtained from the environment condition sensor to determine a validity of the potential medical event.

The group of sensors may include two types of physiological sensors; and the processor may be arranged to correlate between physiological information obtained from the two types of physiological sensors to determine a state of a monitored person.

The portable monitoring unit may include may include, further may include a transmitter; the processor may be arranged to instruct the transmitter to transmit a potential event information in response to a detection of the potential medical event.

The transmitter may be arranged to attempt to transmit the potential event information and to generate a transmission failure indication of the attempt fails; the processor may be arranged to provide an alert that induces a suggestion to change a location of the monitored person or to inform a medical center of the failure by means that differ from the transmitter.

The portable monitoring unit may include a positioning module arranged to determine a position of; the transmitter may be arranged to attempt to transmit the potential event information and to generate a transmission failure indication of the attempt fails; the processor may be arranged to determine a preferred transmission location and generate an alert to a monitored person indicative of the preferred transmission location; a probability of transmission failure from the preferred transmission location may be substantially lower than a probability of a transmission failure at a current location of the monitored person.

The processor may be arranged to activate the transmitter and instruct the transmitter to perform long range transmission of the potential event information in response to a detection of the potential medical event.

The portable monitoring unit may include a transmitter, a primary power source and a secondary power source; the processor may be arranged to prevent a transmission of potential event information during a low power period during which the primary power source may be empty; and to initiate a transmission of potential event information obtained during the low power period.

The processor may be arranged to receive the raw medical information in a continuous manner.

According to an embodiment of the invention a portable monitoring unit is provided. The portable monitoring unit may include a memory module for storing a media clip that may include instructions related to the operation of the monitoring unit, for storing raw medical information, and for storing medical event information; a processor arranged to receive raw medical information from at least one primary sensor and to process the raw medical information and detect potential medical events, to request additional information from at least one secondary sensor in response to a detection of the potential event. The at least one primary sensor and the at least one secondary sensor form a group of sensors; the group of sensors may include an environment condition sensor, a biomechanical sensor and a physiological sensor; and the processor may be arranged to correlate between physiological information obtained from the physiological sensor, biomechanical information obtained from the biomechanical sensor and environment condition information obtained from the environment condition sensor to determine a validity of the potential medical event.

According to an embodiment of the invention a portable monitoring unit is provided. The portable monitoring unit may include a memory module for storing raw medical information, and for storing medical event information; a transmitter, and a processor arranged to receive raw medical information from at least one primary sensor and to process the raw medical information and detect potential medical events, and to instruct the transmitter to transmit a potential event information in response to a detection of the potential medical event; the transmitter may be arranged to attempt to transmit the potential event information and to generate a transmission failure indication if the attempt fails; the processor may be arranged to provide an alert that induces a suggestion to change a location of the monitored person or to inform a medical center of the failure by means that differ from the transmitter.

The portable monitoring unit may include a positioning module arranged to determine a position of a monitored person, the processor may be arranged to determine a preferred transmission location and generate an alert indicative of the preferred transmission location; a probability of transmission failure from the preferred transmission location may be substantially lower than a probability of a transmission failure at a current location of the monitored person.

The portable monitoring unit may include a positioning module arranged to determine a position of; the memory module stores positions of preferred transmission locations and the processor may be arranged to select a preferred transmission location based on the position of and generate an alert indicative of the preferred transmission location; a probability of transmission failure from the preferred transmission location may be substantially lower than a probability of a transmission failure at a current location of the monitored person.

According to an embodiment of the invention a method is provided. The method may include storing in a memory module of a portable monitoring unit, a media clip that may include instructions related to the operation of the monitoring unit, raw medical information and medical event information; receiving raw medical information from at least one primary sensor; and processing the raw medical information to detect potential medical events; displaying the media clip; and performing, by the processor, a potential event related operation in response to a detection of the potential medical event.

The method may include requesting additional information from at least one secondary sensor in response to a detection of the potential event, and processing the additional information to determine a validity of the potential medical event.

The method may include correlating between physiological information obtained from a physiological sensor and biomechanical information obtained from a biomechanical sensor to determine a validity of the potential medical event.

The method may include correlating between physiological information obtained from a physiological sensor and environment condition information obtained from an environment condition sensor to determine a validity of the potential medical event.

The method may include correlating between physiological information obtained from a physiological sensor, biomechanical information obtained from a biomechanical sensor and environment condition information obtained from an environment condition sensor to determine a validity of the potential medical event.

The method may include correlating between physiological information obtained from two types of physiological sensors to determine a state of a monitored person.

The method may include attempting to transmit, by a transmitter of, a potential event information in response to a detection of the potential medical event.

The method may include generating a transmission failure indication if the attempting to transmit fails; and provide an alert that induces a suggestion to change a location of the monitored person or to inform a medical center of the failure by means that differ from the transmitter.

The method may include determining, by a positioning module, a position of the method; attempting to transmit the potential event information; generating a transmission failure indication if the attempting to transmit fails; determining a preferred transmission location; and generating an alert to a monitored person indicative of the preferred transmission location; a probability of transmission failure from the preferred transmission location may be substantially lower than a probability of a transmission failure at a current location of the monitored person.

The method may include activating the transmitter and instructing the transmitter to perform long range transmission of the potential event information in response to a detection of the potential medical event.

The method may include preventing a transmission of potential event information during a low power period during which the primary power source may be empty; and initiating a transmission of potential event information obtained during the low power period.

The method may include receiving the raw medical information in a continuous manner.

According to an embodiment of the invention a method is provided. The method may include storing in a memory module of a portable monitoring unit raw medical information and medical event information; receiving raw medical information from at least one primary sensor; and processing the raw medical information to detect potential medical events; requesting additional information from at least one secondary sensor in response to a detection of the potential event, and processing the additional information to determine a validity of the potential medical event.

The method may include requesting additional information from at least one secondary sensor in response to a detection of the potential event, and processing the additional information to determine a validity of the potential medical event.

The method may include correlating between physiological information obtained from a physiological sensor and biomechanical information obtained from a biomechanical sensor to determine a validity of the potential medical event.

The method may include correlating between physiological information obtained from a physiological sensor and environment condition information obtained from an environment condition sensor to determine a validity of the potential medical event.

The method may include correlating between physiological information obtained from a physiological sensor, biomechanical information obtained from a biomechanical sensor and environment condition information obtained from an environment condition sensor to determine a validity of the potential medical event.

The method may include correlating between physiological information obtained from two types of physiological sensors to determine a state of a monitored person.

The method may include attempting to transmit, by a transmitter of, a potential event information in response to a detection of the potential medical event.

The method may include generating a transmission failure indication if the attempting to transmit fails; and provide an alert that induces a suggestion to change a location of the monitored person or to inform a medical center of the failure by means that differ from the transmitter.

The method may include determining, by a positioning module, a position of the method; attempting to transmit the potential event information; generating a transmission failure indication if the attempting to transmit fails; determining a preferred transmission location; and generating an alert to a monitored person indicative of the preferred transmission location; a probability of transmission failure from the preferred transmission location may be substantially lower than a probability of a transmission failure at a current location of the monitored person.

The method may include activating the transmitter and instructing the transmitter to perform long range transmission of the potential event information in response to a detection of the potential medical event.

The method may include preventing a transmission of potential event information during a low power period during which the primary power source may be empty; and initiating a transmission of potential event information obtained during the low power period.

The method may include receiving the raw medical information in a continuous manner.

According to an embodiment of the invention a method is provided. The method may include storing in a memory module of a portable monitoring unit raw medical information and medical event information; receiving raw medical information from at least one primary sensor; and processing the raw medical information to detect potential medical events; requesting additional information from at least one secondary sensor in response to a detection of the potential event; the at least one primary sensor and the at least one secondary sensor form a group of sensors; and correlating between physiological information obtained from a physiological sensor of the group of sensors, biomechanical information obtained from a biomechanical sensor of the group of sensors, and environment condition information obtained from an environment condition sensor of the group of sensors to determine a validity of the potential medical event.

According to an embodiment of the invention a method is provided. The method may include storing in a memory module of a portable monitoring unit raw medical information and medical event information; receiving of raw medical information from at least one primary sensor; and processing the raw medical information to detect potential medical events; requesting additional information from at least one secondary sensor in response to a detection of the potential event, processing the additional information to determine a validity of the potential medical event; and attempting to transmit, by a transmitter of, a potential event information in response to a detection of the potential medical event.

The method may include generating a transmission failure indication if the attempting to transmit fails; and provide an alert that induces a suggestion to change a location of the monitored person or to inform a medical center of the failure by means that differ from the transmitter.

The method may include determining, by a positioning module, a position of the method; attempting to transmit the potential event information; generating a transmission failure indication if the attempting to transmit fails; determining a preferred transmission location; and generating an alert to a monitored person indicative of the preferred transmission location; a probability of transmission failure from the preferred transmission location may be substantially lower than a probability of a transmission failure at a current location of the monitored person.

### BRIEF DESCRIPTION OF THE DRAWING

The foregoing and other objects, features, and advantages of the present invention will become more apparent from the following detailed description when taken in conjunction with the accompanying drawings. In the drawings, similar reference characters denote similar elements throughout the different views, in which:

FIG. 1 illustrates a portable monitoring unit according to an embodiment of the invention;

FIG. 2 illustrates a portable monitoring unit according to another embodiment of the invention;

FIG. 3 illustrates a group of sensors that includes a physiological sensor and a biomechanical sensor according to an embodiment of the invention;

FIG. 4 illustrates a group of sensors that includes an environment condition sensor and a physiological sensor according to an embodiment of the invention;

FIG. 5 illustrates a group of sensors that includes an environment condition sensor, a biomechanical sensor and a physiological sensor according to an embodiment of the invention;

FIG. 6 illustrates a group of sensors that includes two types of physiological sensors according to an embodiment of the invention;

FIG. 7 illustrates a portable monitoring system that also includes a positioning module that is arranged to determine a position of the monitored person according to an embodiment of the invention;

FIG. 8 illustrates a portable monitoring system that includes a primary power source and a secondary power source according to an embodiment of the invention;

FIG. 9 illustrates a portable monitoring unit according to an embodiment of the invention;

FIG. 10 illustrates a portable monitoring unit according to an embodiment of the invention;

FIG. 11 illustrates a portable monitoring unit, additional sensors and a cellular phone according to an embodiment of the invention;

FIG. 12 illustrates a method according to an embodiment of the invention;

FIG. 13 illustrates various potential event related operations according to various embodiments of the invention; and

FIG. 14 illustrates a method according to an embodiment of the invention.

### DETAILED DESCRIPTION OF THE DRAWING

A portable monitoring unit is provided. It can be easily operated by a monitored user as it can operate automatically with minimal intervention. Operating the portable monitoring unit can be eased by playing video clip that includes instructions related to the operation of the monitoring unit.

The portable monitoring unit can process raw medical data and is it detects a potential medical event it can request information from other sensors and correlate between information received from different types of sensors in order to validate the potential medical event and determine that an (actual) medical event occurred.

The portable monitoring unit can transmit information to a medical center in response to an occurrence of a medical event and it attempts to transmit such information fails may induce the monitored user to either improve the chances of succeeding in the transmission attempt or contact a medical center by using a device that differs from the portable monitoring unit. Thus, if a medical event is detected the chances of providing a report to the medical center increase. The portable monitored unit can display to the monitored person selected medical information and the monitored person may report the selected medical information to the medical center.

The term medical center refers to any computer, storage unit, or facility that may store medical information so that the stored medical information may be accessed (in real time or in off-line) by medical personal such as doctors, nurses and the like.

The term medical information refers to any information that may describe the medical status of a monitored person. It may include, for example, heart related information, blood related information, or any information related to one or more organs of a monitored person.

The term medical event may relate to any event that can affect (for example have a negative impact on) or can measure the health of a monitored person. Non-limiting examples may include a deviation of a physiological parameter from an allowed range such as high blood pressure, low blood pressure, high glucose level, low oxygen levels. A medical event can also include a measurements itself or an absence of a measurement.

The term primary sensor may relate to a sensor that may provide information that may cause a controller to request information from another sensor (termed a secondary sensor). The classification of a sensor as a primary sensor or a secondary sensor can change over time and per task or application. Thus, a primary sensor can become a secondary sensor and vice verse.

The term wellness status may relate to an evaluation about the health of a monitored person. The evaluation can be responsive to medical events, potential medical events, measurements made by sensors, lack of medical events and the like. The wellness status can have a wellness status value (or more than one value) that provides an indication about the health of the monitored person.

In this specification various alerts or instructions are described as aimed to the monitored person. It is noted that these alerts or instructions can be received by persons that differ from the monitored person and these other persons can react to these alerts or instructions by operating the portable monitoring unit, by calling a medical center, and the like.

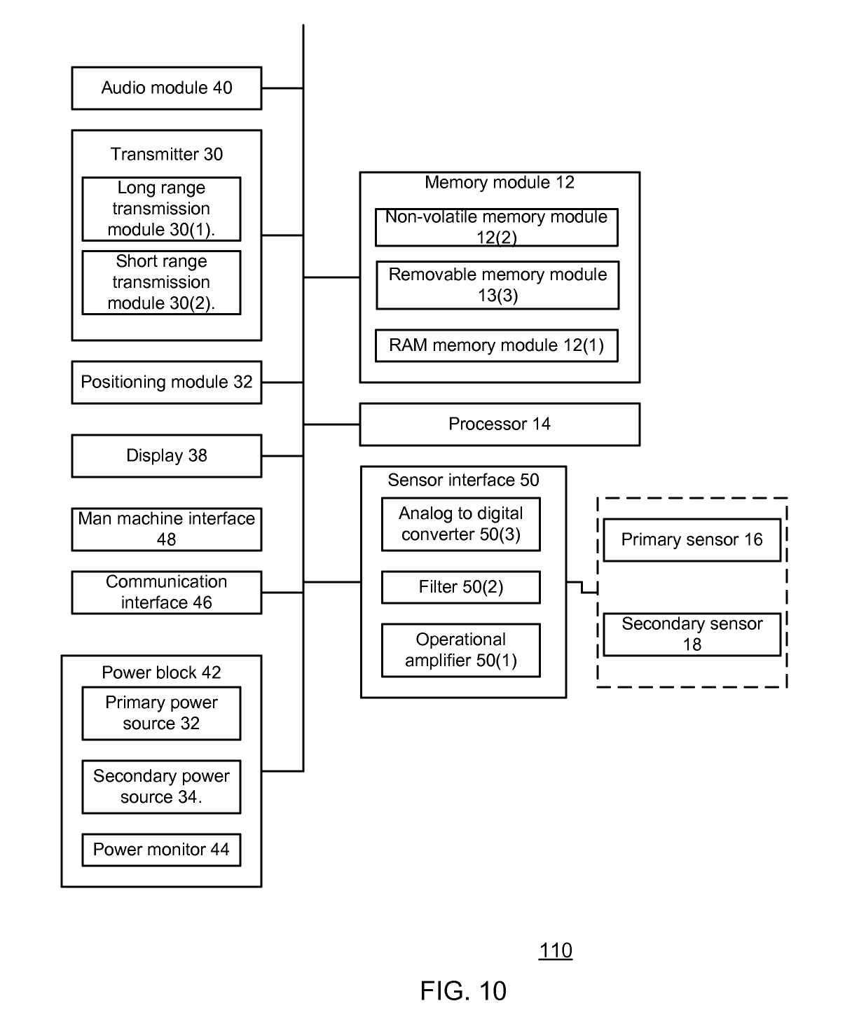

It is noted that in the following figures various components may be missing—for simplicity of explanation. For example either one of the portable monitoring units may include a power source although an example of a power source is included only in only few figures. Yet for another example, a sensor interface is illustrated in FIG. 10 although each portable monitoring unit may have one or more sensor interfaces.

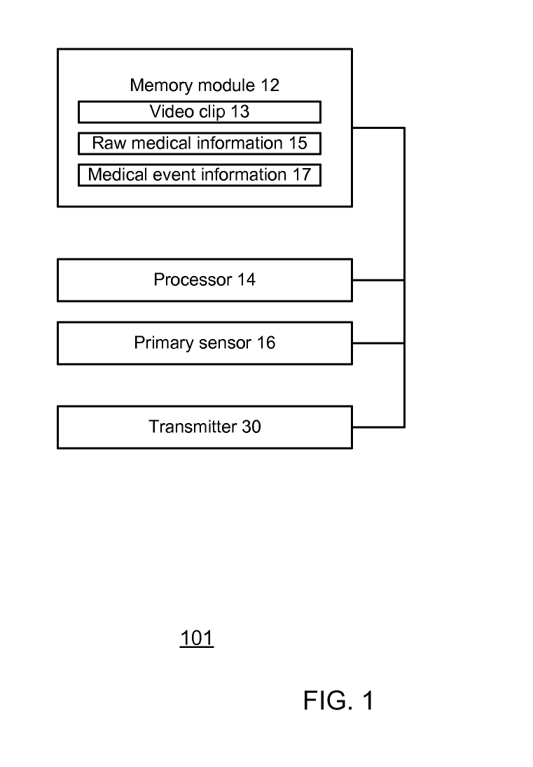

FIG. 1 illustrates a portable monitoring unit 101 according to an embodiment of the invention. The portable monitoring unit 101 is portable in the sense that it is compact and light and can be easily worn or otherwise carried by a monitored person. It may weigh below 1 kg or below 400 grams and be about the size of a cigarette pack.

The portable monitoring unit 10 may include a memory module 12 for storing a media clip 13 that may include instructions related to the operation of the monitoring unit, for storing raw medical information 15, and for storing medical event information 17; a processor 14 arranged to receive raw medical information from at least one primary sensor 16 and to process the raw medical information 15 and detect potential medical events, to trigger a display of the media clip 13 and to perform a potential event related operation in response to a detection of the potential medical event.

The primary and secondary sensors can be integrated in the portable monitoring unit 10. The portable monitoring unit 10 can include sensors such as a sugar sensor, a heart rate sensor, an ECG sensor, a temperature sensor, a radar based sensor (such as sensors that include Ultra Wide Band antennas for radar based sensing) and the like.

The portable monitoring unit 10 can receive information from other sensors and can correlate between data provided from the other sensors and the sensors included in the portable monitoring unit.

The portable monitoring unit 10 can be a hand held device, a personal data accessory (or included in such a PDA), a smart phone, a tablet and the like (or integrated within). Thus, primary and secondary sensors can be included in a component such as a PDA, a smart phone or a tablet but may also be located outside such components and communicate with these components.

The portable monitoring unit 10 can perform a test and save the results of the test. It can transmit medical information to a remote station (such as a medical center) in one or more manners—according to a predefined timing schedule, at predefined intervals, in response to one or more events, in response to sensed physiological parameters that exceed predefined thresholds or are below predefined thresholds, per hour of a day, per requests or definition of a patient or a doctor.

According to an embodiment of the invention the portable monitoring unit 10 can transmit information (directly or indirectly) to a third party such that the third part can receive a status of the monitored person. Thus, if a certain test is scheduled then the third party can receive an indication that the test succeeded, failed or was not conducted at all.

According to various embodiments of the invention the processor 14 can send to a display the raw medical information.

According to various embodiments of the invention a validity of a wellness status can be evaluated by a processor. The validity of a wellness status can involve correlating between information obtained from sensors from multiple types such as a physiological sensor, a timer, an environment condition sensor, a real time clock, and the like. A wellness status can trigger a transmission of information to a third party.

The portable monitoring unit 101 also includes a transmitter 30. The processor 14 may be arranged to instruct the transmitter to transmit potential event information in response to a detection of the potential medical event value or to a predefined wellness status value (for example—a wellness status that may indicate that the wellness of the monitored person is below a desired threshold). The transmission of the potential medical event information can be triggered per a request of the monitored person or a third party (by instructing the portable monitoring unit 101 or by using a remote device).

According to an embodiment of the invention the portable monitoring unit 101 can include a still camera, a video camera and the like. The images acquired from such cameras can be processed by the processor 14, stores and, additionally or alternatively, be transmitted by the transmitter 30. The images can be processed to detect motion and provide biophysical information that can be correlated with information from other sensors.

At least one sensor can communicate with the portable monitor unit 101 using a short range receiver or transceiver such as a short range radio frequency receiver or transceiver.

Potential event information can be transmitted in predefined times, in response to a request from a monitored person to transmit such information, or in response to a request from a third party to transmit such information.

According to an embodiment of the invention the transmitter 30 may be arranged to transmit an indication about a completion of a measurement by the portable monitoring unit 101 to a third party. Additionally or alternatively, the transmitter 30 can transmit an indication, to a third party, about an incompletion of a measurement by the portable monitoring unit 101. The processor 30 can instruct the transmitter 30 to send such indications. It is noted that an incompletion of a measurement can include an event in which the measurement did not start and an event in which the measurement started by was not completed.

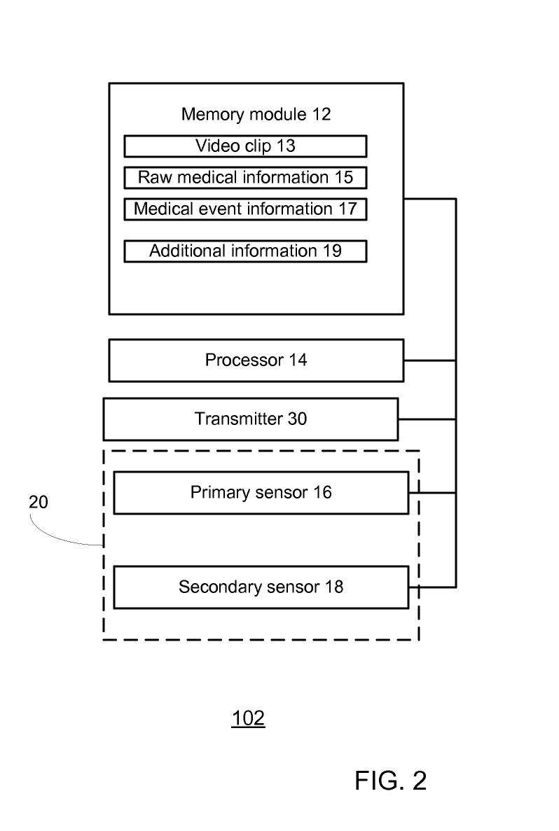

FIG. 2 illustrates a portable monitoring unit 102 according to another embodiment of the invention. The portable monitoring unit 102 may be coupled (or may include) to at least one secondary sensor 18. The processor 14 may be arranged to request additional information (illustrated as box 19 that is stored in memory module 12) from at least one secondary sensor 18 in response to a detection of the potential event, and to process the additional information 19 to determine a validity of the potential medical event. The at least one primary sensor 16 and the at least one secondary sensor 18 may form a group of sensors 20.

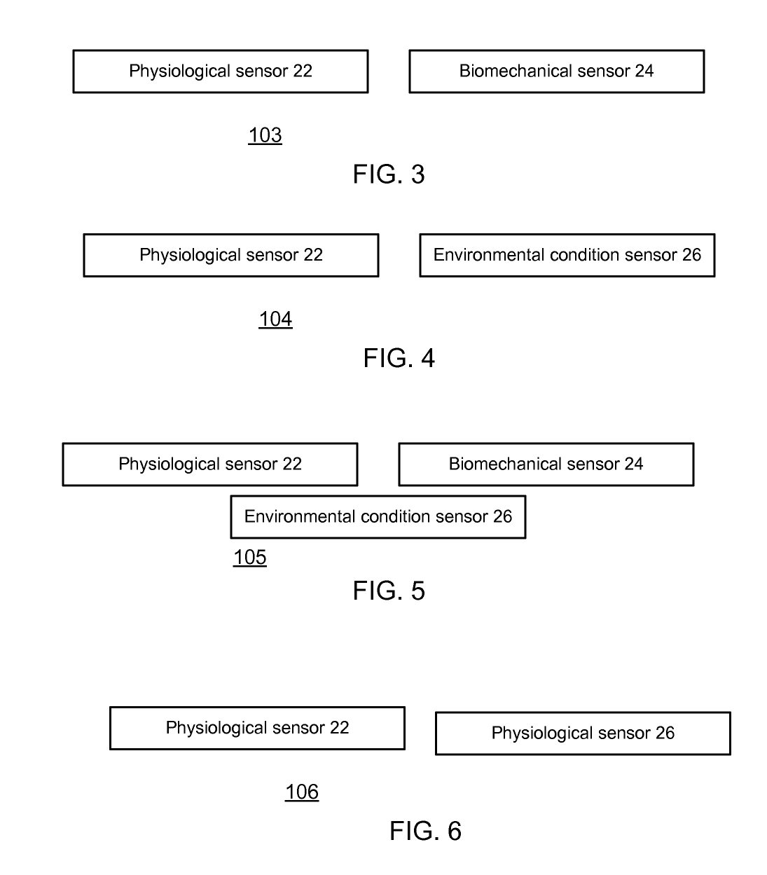

FIG. 3 illustrates a group of sensors 103 that includes a physiological sensor 22 and a biomechanical sensor 24. The processor 14 of FIG. 1 or 2 may be arranged to correlate between physiological information obtained from the physiological sensor 22 and biomechanical information obtained from the biomechanical sensor 24 to determine a validity of the potential medical event.

FIG. 4 illustrates a group of sensors 104 that includes an environment condition sensor 26 and a physiological sensor 22. The processor 14 may be arranged to correlate between physiological information obtained from the physiological sensor 22 and environment condition information obtained from the environment condition sensor 26 to determine a validity of the potential medical event.

FIG. 5 illustrates a group of sensors 105 that includes an environment condition sensor 26, a biomechanical sensor 24 and a physiological sensor 22. The processor 14 may be arranged to correlate between physiological information obtained from the physiological sensor 22, biomechanical information obtained from the biomechanical sensor 24 and environment condition information obtained from the environment condition sensor 26 to determine a validity of the potential medical event.

FIG. 6 illustrates a group of sensors 106 that includes two types of physiological sensors 22 and 28. The processor 14 may be arranged to correlate between physiological information obtained from the two types of physiological sensors 22 and 28 to determine a state of a monitored person.

According to an embodiment of the invention the transmitter 30 may be arranged to attempt to transmit the potential event information and to generate a transmission failure indication of the attempt fails. The transmission failure indication is sent to the processor 14 that may be arranged to provide an alert (visual, audio or a combination thereof) that induces a suggestion to change a location of the monitored person or to inform a medical center of the failure by means that differ from the transmitter.

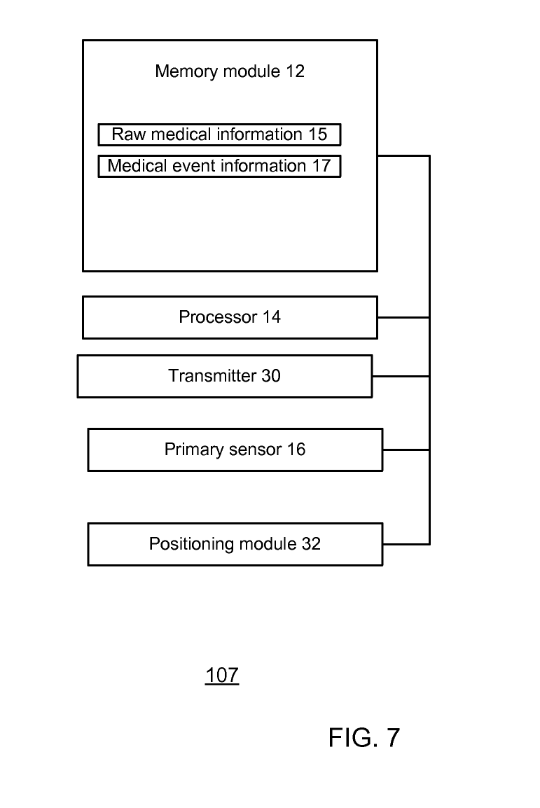

FIG. 7 illustrates a portable monitoring system 107 that also includes a positioning module 32 that is arranged to determine a position of the monitored person. The transmitter 30 may be arranged to attempt to transmit the potential event information and to generate a transmission failure indication of the attempt fails. The processor 14 may be arranged to determine a preferred transmission location and generate an alert to a monitored person indicative of the preferred transmission location. The preferred transmission location is expected to be better suited for transmission that the current location of the monitored person. The memory module 12 may receive and store information about preferred transmission locations and the processor may retrieve this information and select a preferred transmission location that is the closest to the current location of the monitored person or otherwise easy to arrive to. The location information can be transmitted together with other types of information (such as medical information transmittal) or separately.

In order to reduce the power consumption of the portable monitoring unit the processor 14 may be arranged to activate the transmitter 30 only when there is a need to transmit potential event information.

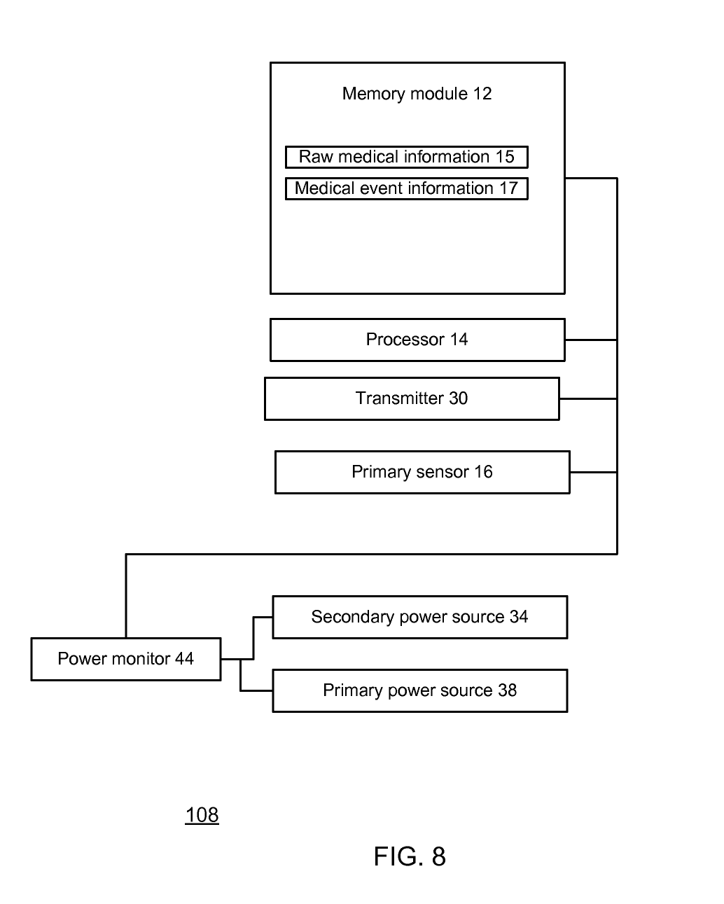

FIG. 8 illustrates a portable monitoring system 108 that includes a primary power source 38 and a secondary power source 34. The processor 14 may be arranged to prevent a transmission of potential event information during a low power period during which the primary power source 38 may be empty. The emptiness of the primary power source 38 may be monitored by the processor or by another entity such as a power monitor 44. The power monitor 44, the primary power source 38 and the secondary power source 34 form a power module 42. The primary power source 38 can be recharged or replaced while the secondary power source powers the portable monitoring unit. The processor 14 may initiate a transmission of potential event information obtained during the low power period.

Any of the mentioned above portable monitoring units may be arranged to operate in a “holter” or continuous mode in which raw medical information is constantly obtained and may act in an “analysis” mode when the processor 14 detects a potential medical event.

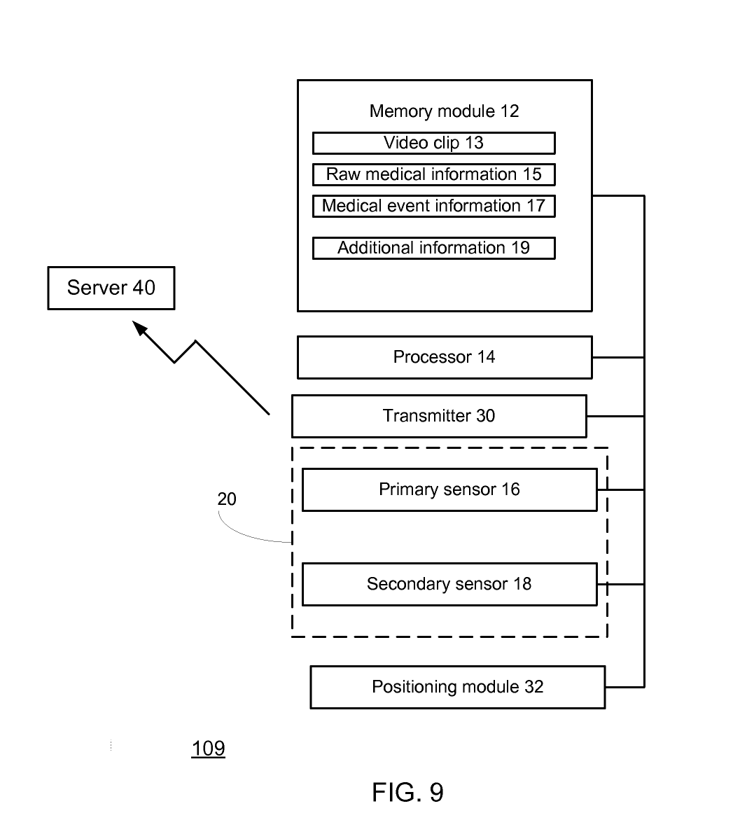

FIG. 9 illustrates a portable monitoring unit 109 according to an embodiment of the invention. The portable monitoring unit 109 may include a memory module 12 for storing a media clip that may include instructions related to the operation of the monitoring unit, for storing raw medical information, and for storing medical event information; a processor 14 arranged to receive raw medical information from at least one primary sensor 16 and to process the raw medical information and detect potential medical events, to request additional information from at least one secondary sensor 18 in response to a detection of the potential event, and to process the additional information to determine a validity of the potential medical event. T

The at least one primary sensor 16 and the at least one secondary sensor 18 form a group of sensors 20. The group of sensors can include any of the sensors illustrated in either one of FIG. 3-FIG. 6. The processor 14 can correlate between information provided by any of such sensors.

The portable monitoring unit 109 also includes a transmitter 30 and a positioning module 32. The processor 14 may be arranged to instruct the transmitter 30 to transmit potential event information in response to a detection of the potential medical event or based on predefined times, or based on a request of the monitored person to transmit such information.

The positioning module 32 may be arranged to determine a position of the monitored person (of the portable monitoring unit 109). The determination can be based on the Global Positioning System (GPS), can be based on mobile network technology (triangulation based on reception of cells of the mobile network) or any other positioning system.

The determination of the location of the monitored person can assist in various aspects. For example, movements of the monitored person (such as running, walking, sitting in a vehicle) can be monitored by the positioning module 32. Thus, the positioning module 32 can be used as a biomechanical sensor. Additionally or alternatively, the positioning module 32 can monitor the location of the monitored person and can send this information to the processor 14 that may, in turn, suggest changing the location of the monitored person in order to increase the chances of success in transmission attempts from the portable monitoring unit 109. The portable monitoring unit 109 can receive information about preferred transmission points in various manners. For example, it can request to obtain such information over the network, by transmitting a request over a wireless or wired network.

A server or other data providing entity (such as server 40 of FIG. 9) can send information about preferred transmission points—in response to the request. Local information about preferred transmission points can be sent to the portable monitoring unit 109 based upon the position of the monitored person and may include a set of preferred transmission points within a certain range or area that includes the current location of the monitored person. This is not necessarily so and preferred transmission points that belong to a large area can be sent to the portable monitoring unit 109 regardless of the location of the monitored portion.

According to an embodiment of the invention the server or a data providing entity can also request information from the portable monitoring unit 109 such as raw medical data, raw data from various sensors, processed information and the like. The server or data providing entity can update the software of the portable monitoring unit 109, can change settings, and trigger measurements by the portable monitoring unit 109.

The portable monitoring unit 109 can send a request to obtain new preferred transmission points information each time period, in response to the location (or a change in a location) of the monitored person and the like.

The information about the location of the preferred transmission points can be obtained in one or more manners. For example, a mobile network operator can provide such information. The transmissions of the portable monitoring unit 109, and additionally or alternatively, the signals received by the portable monitoring unit 109 can be monitored. Higher transmission success rates and stronger received signals can provide an indication of the preferred transmission locations.

The transmitter 30 may be arranged to attempt to transmit the potential event information and to generate a transmission failure indication of the attempt fails.

The processor 14 may be arranged to determine a preferred transmission location and generate an alert to a monitored person indicative of the preferred transmission location. The preferred transmission location is expected to be better suited for transmission that the current location of the monitored person. The memory module 12 may receive and store information about preferred transmission locations and the processor may retrieve this information and select a preferred transmission location that is the closest to the current location of the monitored person or otherwise easy to arrive to.

According to an embodiment of the invention the portable monitoring unit 109 can be loaded with preferred transmission point information before being provided to the monitored person.

According to an embodiment of the invention the monitored person can provide information about his expected route and the processor 12 can elect preferred transmission points that are proximate to that route.

FIG. 10 illustrates a portable monitoring unit 110 according to an embodiment of the invention.

The portable monitoring unit 110 includes processor 14, memory module 12, display 38, audio module 40 that may include a speaker, a amplifier, a microphone and a codec, a power block 42, a transmitter 30 that may include a long range transmission module 30(1), and additionally or alternatively, a short-range transmission module 30(2), a communication interface 46, a man machine interface (MMI) 48 and a sensor interface 50 that may be coupled to multiple primary sensors 16. One or more of these sensors can be a part of the portable monitoring unit 110 or be coupled to the portable monitoring unit 110. It is noted that the sensor interface 50 can be coupled to secondary sensors 18. A sensor interface can be allocated per one or more sensors.

The memory module 12 may include at least one out of a RAM memory module 12(1), a non-volatile memory module 12(2) and a removable memory module 13(3) that may include a flash memory card, a secure digital memory card. Each type of information mentioned in the specification (for example—raw medical information 15, media clip 13, medical event information 17, additional information 19) can be stored in one or more of these modules 13(1), 13(2) and 13(3).

The display 38 can be used to display a video clip to the monitored person, to display selected medical information—including selected medical information that should be reported by the monitored person to a medical center or another third party if the transmitter 30 fails to transmit event related information or potential event related information to the medical center. Non-limiting examples of a third party can be a family member, a neighbor, an insurance company, a medical staff member, an ambulance.

The communication interface 46 can receive instructions, medical parameters for evaluating potential medical events, for validating medical events, and the like. It can be JTAG compliant, USB compliant, SPI compliant and the like. The MMI 48 can be a keyboard, a touch screen or can be audio based. The power module 42 supplied power from either one of the primary power source 32 or the secondary power source 34. The emptiness level of the primary power source 32 can be monitored by a power monitor 44 that may decide that there is a need to replace or recharge a power source (out of 32 and 34) when their power level reaches a predefined threshold. The power monitor 44 may also select a power source that should provide the power to the other components of the portable monitoring unit 110.

The long range transmission module 30(1) can communicate using at least one known method or protocol such as GSM, GPRS, CDMA, UMTS, WI-Max, Wi-Fi, UWB and the like. The short-range transmission module 30(2) can communicate using at least one known method, protocol or radiation such as Bluetooth, Radio Frequency, Infrared, and the like.

The sensor interface 50 can include an operational amplifier 50(1) that receives detection signals from one or more primary and/or secondary sensors, a filter 50(2) and an analog to digital converter 50(3). The output of the analog to digital converter 50(3) provides raw medical data. Alternatively, the output of the analog to digital converter 50(3) can be sampled and/or additionally processed to provide the raw medical data.

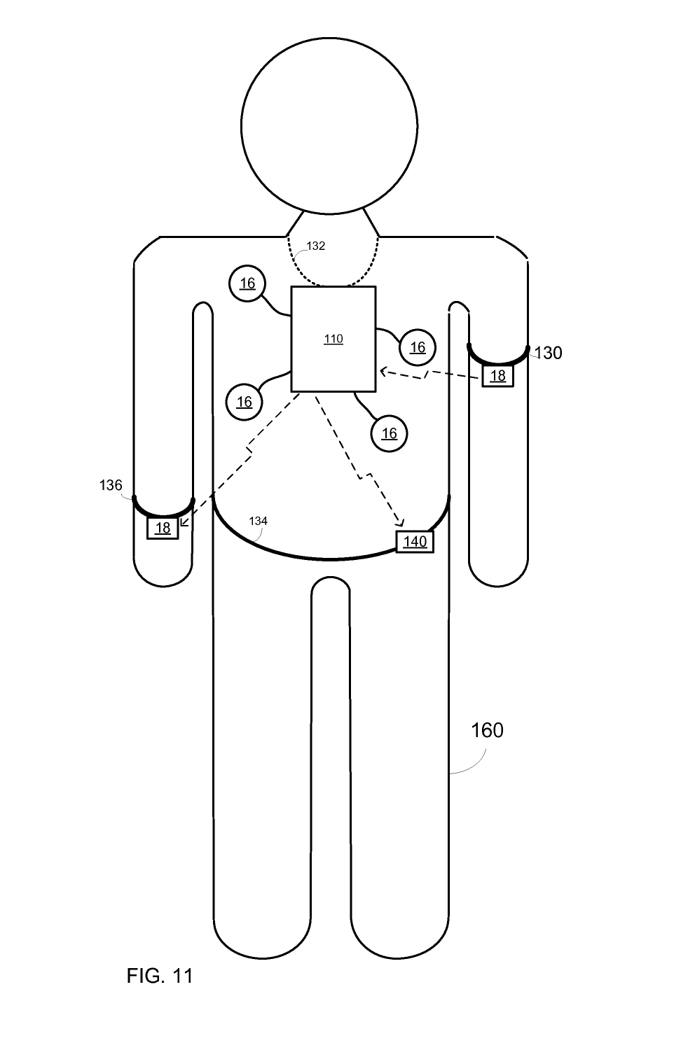

FIG. 11 illustrates a portable monitoring unit 110, additional sensors and a cellular phone according to an embodiment of the invention.

The portable monitoring unit 110 is attached to a strip 132 that is worn around the neck of a monitored person 160. The portable monitoring unit 110 is coupled by electrodes to primary sensors 16, and communicated by short range transmission with secondary detectors 18 that are worn on a hand cuff 130 and a wristband 136. The portable monitoring unit 110 also wirelessly communicates with a mobile phone 140. The portable monitoring unit 110 can wirelessly relay information to the mobile phone when, for example, the portable monitoring unit 110 is not able to communicate with a medical center. The mobile phone can attempt to transmit the information to the medical center and may inform the portable monitoring unit 110 about a success or a failure in this attempt. In case of a failure the portable monitoring unit 110 may, for example, ask the monitored person 160 to change his position, and in case of success the portable monitoring unit 110 can decide not to re-transmit the relayed information to the medical center although it may re-transmit the medical information to the medical center.

It is noted that one or more primary sensor may wirelessly communicate with the portable monitoring unit 110 and that one or more secondary sensor 18 may be connected by wire to the portable monitoring unit 110.

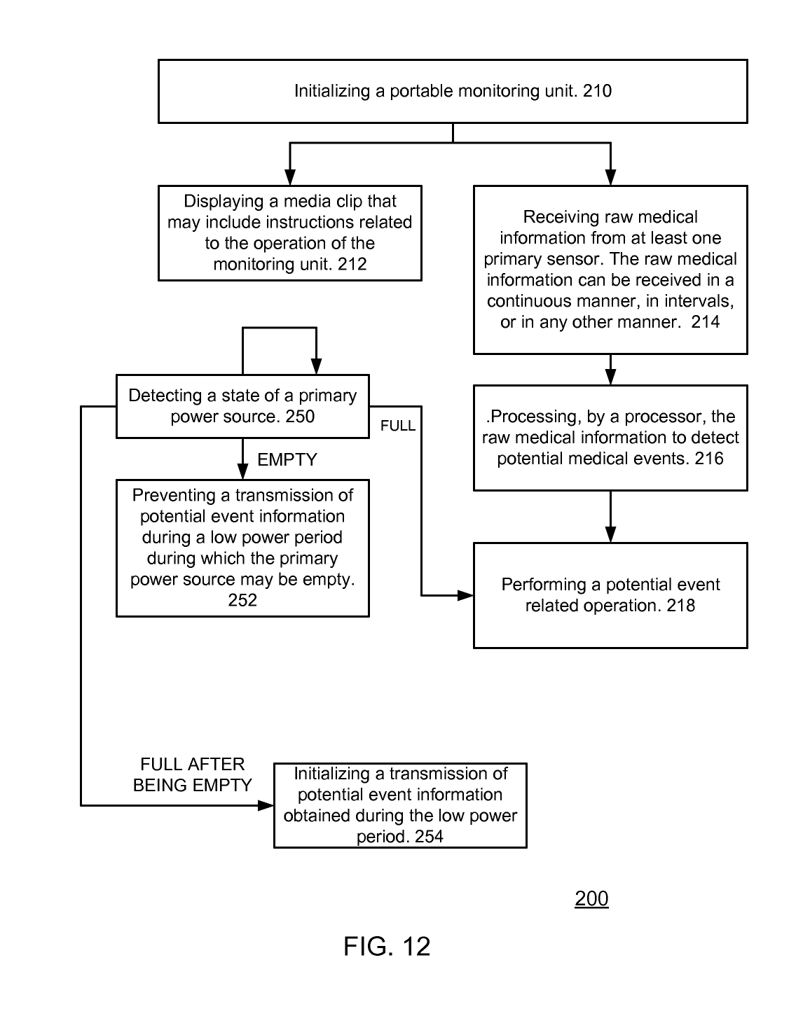

FIG. 12 illustrates a method 200 according to an embodiment of the invention.

Method 200 may stars by an initialization (210) of a portable monitoring unit. The initialization may include resetting the portable monitoring unit so that it will not store raw physiological information or processed medical information. The initialization may include configuring the portable monitoring unit by providing preferred transmission points information, medical event characteristics such as values of raw medical information that can indicate that a potential medical event occurred or that a (real) medical event occurred. The initialization can also include setting various parameters such as sampling rate of the raw medical information, algorithms that are to be used to detect potential medical events and validate medical events.

The initialization 210 may include storing, in a memory module of a portable monitoring unit, a media clip that may include instructions related to the operation of the monitoring unit.

The initialization 210 may be followed by a display (212) of the media clip. The display can be triggered by a request from the monitored person, can be triggered at the first time the portable monitoring unit is being used or by any other trigger.

Additionally or alternatively, the initialization or the display of the clip can be followed by a reception (214) of raw medical information from at least one primary sensor. The raw medical information can be received in a continuous manner, in intervals, or in any other manner. The reception can include receiving raw medical information from one or more sensors, storing the raw medical information in a memory module and retrieving the raw medical information from the memory module. The reception may include providing the raw medical information to the memory module and to the processor in parallel or in a pipelined manner.

The reception of raw information can be followed by a processing (216), by the processor, the raw medical information to detect potential medical events.

It is noted that the processing of raw medical information can be executed in parallel to a reception of other raw medical information. The reception of the raw medical information can occur constantly and even during long periods (days, weeks and months) and the processing of the raw medical information can be executed during these periods.

In response to a detection of a potential medical event, the processor may perform (218) a potential event related operation.

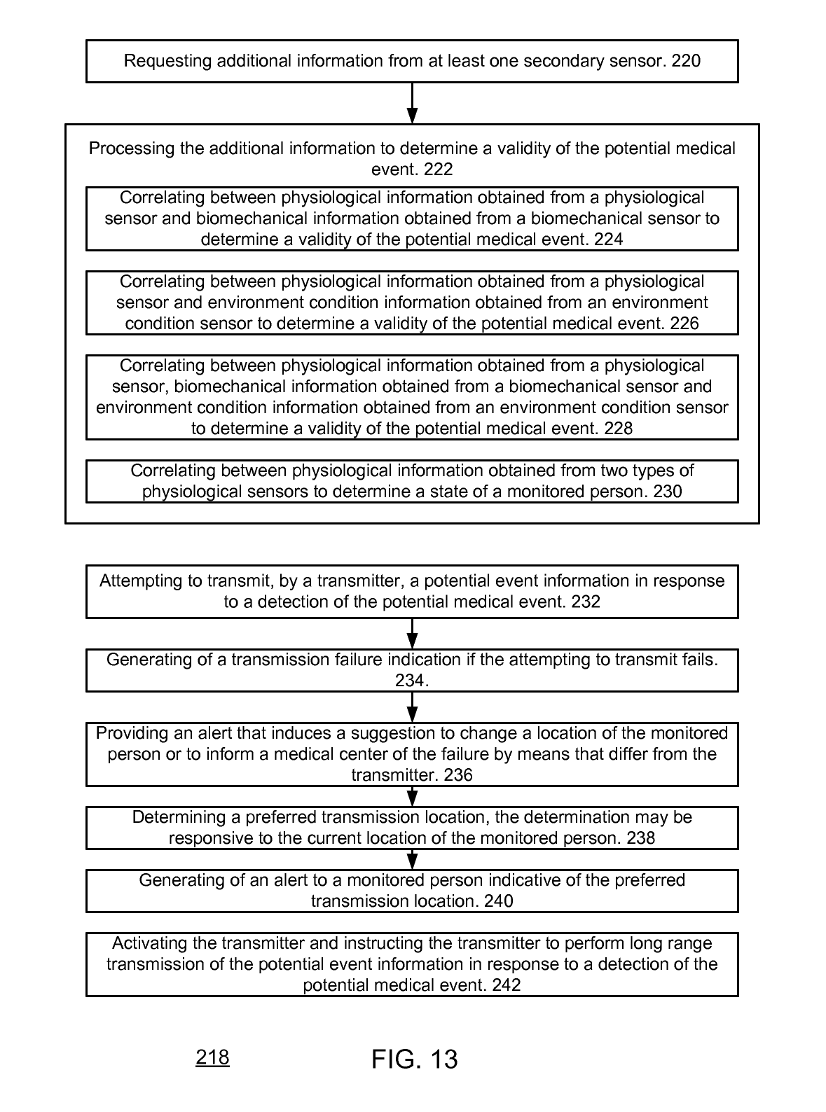

FIG. 13 illustrates various potential event related operations according to various embodiments of the invention. The performing of a potential event related operation may include at least one of the following or a combination thereof: (a) a request (220) for additional information from at least one secondary sensor, (b) a processing (222) of the additional information to determine a validity of the potential medical event, (c) a correlation (224) between physiological information obtained from a physiological sensor and biomechanical information obtained from a biomechanical sensor to determine a validity of the potential medical event, (d) a correlation (226) between physiological information obtained from a physiological sensor and environment condition information obtained from an environment condition sensor to determine a validity of the potential medical event; (e) a correlation (228) between physiological information obtained from a physiological sensor, biomechanical information obtained from a biomechanical sensor and environment condition information obtained from an environment condition sensor to determine a validity of the potential medical event; (f) a correlation (230) between physiological information obtained from two types of physiological sensors to determine a state of a monitored person; (g) an attempt (232) to transmit, by a transmitter, a potential event information in response to a detection of the potential medical event; (h) a generation (234) of a transmission failure indication if the attempting to transmit fails; (i) a provision (236) of an alert that induces a suggestion to change a location of the monitored person or to inform a medical center of the failure by means that differ from the transmitter; (j) a determination (238) of a preferred transmission location, the determination may be responsive to the current location of the monitored person; (k) a generation (240) of an alert to a monitored person indicative of the preferred transmission location; a probability of transmission failure from the preferred transmission location may be substantially lower than a probability of a transmission failure at a current location of the monitored person; (l) an activation (242) of the transmitter and instructing the transmitter to perform long range transmission of the potential event information in response to a detection of the potential medical event.

The method may include a detection (250) of a state of a primary power source; a prevention (252) of a transmission of potential event information during a low power period during which the primary power source may be empty; and an initiation (254) of a transmission of potential event information obtained during the low power period.

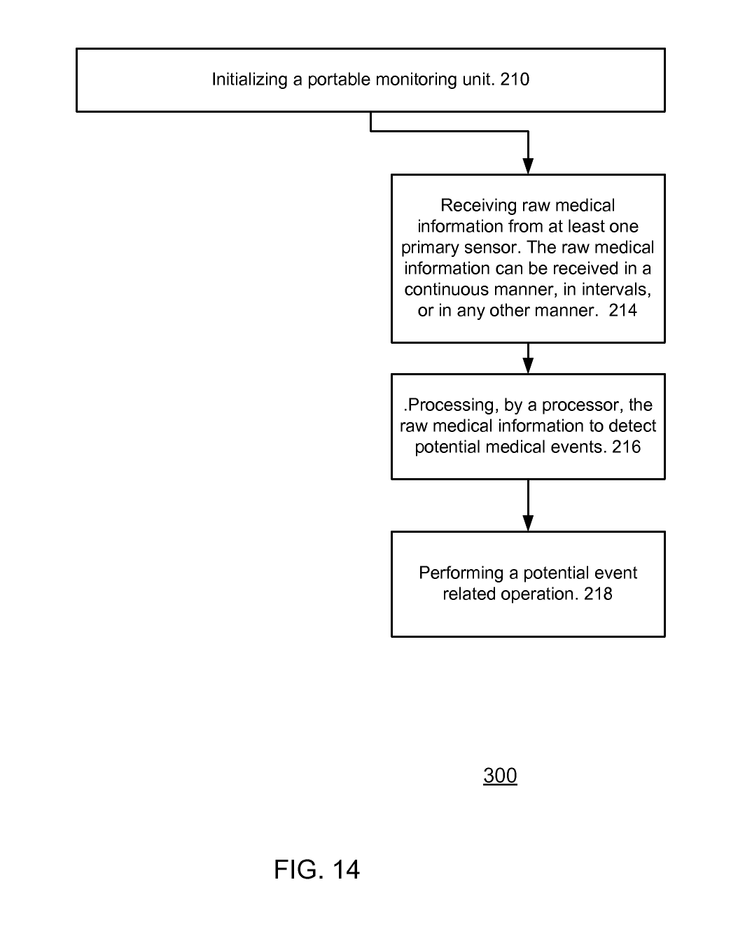

FIG. 14 illustrates a method 300 according to an embodiment of the invention.

Method 300 may stars by an initialization (210) of a portable monitoring unit. The initialization may include resetting the portable monitoring unit so that it will not store raw physiological information or processed medical information. The initialization may include configuring the portable monitoring unit by providing preferred transmission points information, medical event characteristics such as values of raw medical information that can indicate that a potential medical event occurred or that a (real) medical event occurred. The initialization can also include setting various parameters such as sampling rate of the raw medical information, algorithms that are to be used to detect potential medical events and validate medical events.

The initialization 210 may include storing, in a memory module of a portable monitoring unit, a media clip that may include instructions related to the operation of the monitoring unit.

The initialization 210 may be followed by a reception (214) of raw medical information from at least one primary sensor. The raw medical information can be received in a continuous manner, in intervals, or in any other manner. The reception can include receiving raw medical information from one or more sensors, storing the raw medical information in a memory module and retrieving the raw medical information from the memory module. The reception may include providing the raw medical information to the memory module and to the processor in parallel or in a pipelined manner.

The reception of raw information can be followed by a processing (216), by the processor, the raw medical information to detect potential medical events.

In response to a detection of a potential medical event, the processor may perform (218) a potential event related operation. Non-limiting examples of a potential event related operation are illustrated in FIG. 13.

The present invention can be practiced by employing conventional tools, methodology, and components. Accordingly, the details of such tools, component, and methodology are not set forth herein in detail. In the previous descriptions, numerous specific details are set forth, in order to provide a thorough understanding of the present invention. However, it should be recognized that the present invention might be practiced without resorting to the details specifically set forth.

Only exemplary embodiments of the present invention and but a few examples of its versatility are shown and described in the present disclosure. It is to be understood that the present invention is capable of use in various other combinations and environments and is capable of changes or modifications within the scope of the inventive concept as expressed herein.

We claim:

1. A portable monitoring unit, comprising:
a memory module for storing a media clip that comprises instructions related to the operation of the monitoring unit, for storing raw medical information, and for storing medical event information; and
a processor arranged to receive raw medical information from at least one primary sensor and to process the raw medical information and detect potential medical events, to trigger a display of the media clip and to perform a potential event related operation in response to a detection of the potential medical event.

2. The portable monitoring unit according to claim 1, wherein the processor is arranged to request additional information from at least one secondary sensor in response to a detection of the potential event, and to process the additional information to determine a validity of the potential medical event, wherein the at least one primary sensor and the at least one secondary sensor form a group of sensors.

3. The portable monitoring unit according to claim 2, wherein the group of sensors comprises a physiological sensor and a biomechanical sensor; and wherein the processor is arranged to correlate between physiological information obtained from the physiological sensor and biomechanical information obtained from the biomechanical sensor to determine a validity of the potential medical event.

4. The portable monitoring unit according to claim 2, wherein the group of sensors comprises an environment condition sensor and a physiological sensor; and wherein the processor is arranged to correlate between physiological information obtained from the physiological sensor and environment condition information obtained from the environment condition sensor to determine a validity of the potential medical event.

5. The portable monitoring unit according to claim 2, wherein the group of sensors comprises an environment condition sensor, a biomechanical sensor and a physiological sensor; and wherein the processor is arranged to correlate between physiological information obtained from the physiological sensor, biomechanical information obtained from the biomechanical sensor and environment condition information obtained from the environment condition sensor to determine a validity of the potential medical event.

6. The portable monitoring unit according to claim 2, wherein the group of sensors comprises two types of physiological sensors; and wherein the processor is arranged to correlate between physiological information obtained from the two types of physiological sensors to determine a state of a monitored person.

7. The portable monitoring unit according to claim 1, further comprising a transmitter; wherein the processor is arranged to instruct the transmitter to transmit a potential event information in response to a detection of the potential medical event.

8. The portable monitoring unit according to claim 7, wherein the transmitter is arranged to attempt to transmit the potential event information and to generate a transmission failure indication of the attempt fails; wherein the processor is arranged to provide an alert that induces a suggestion to change a location of the monitored person or to inform a medical center of the failure by means that differ from the transmitter.

9. The portable monitoring unit according to claim 7, comprising a positioning module arranged to determine a position of the portable monitoring unit; wherein the transmitter is arranged to attempt to transmit the potential event information and to generate a transmission failure indication of the attempt fails; wherein the processor is arranged to determine a preferred transmission location and generate an alert to a monitored person indicative of the preferred transmission location; wherein a probability of transmission failure from the preferred transmission location is substantially lower than a probability of a transmission failure at a current location of the monitored person.

10. The portable monitoring unit according to claim 7, wherein the processor is arranged to activate the transmitter and instruct the transmitter to perform long range transmission of the potential event information in response to a detection of the potential medical event.

11. The portable monitoring unit according to claim 1, comprising a transmitter, a primary power source and a secondary power source; wherein the processor is arranged to prevent a transmission of potential event information during a low power period during which the primary power source is empty; and to initiate a transmission of potential event information obtained during the low power period.

12. The portable monitoring unit according to claim 1, wherein the processor is arranged to receive the raw medical information in a continuous manner.

13. A portable monitoring unit, comprising:
a memory module for storing a media clip that comprises instructions related to the operation of the monitoring unit, for storing raw medical information, and for storing medical event information; and
a processor arranged to receive raw medical information from at least one primary sensor and to process the raw medical information and detect potential medical events, to request additional information from at least one secondary sensor in response to a detection of the potential event, and to process the additional information to determine a validity of the potential medical event, wherein the at least one primary sensor and the at least one secondary sensor form a group of sensors.

14. The portable monitoring unit according to claim 13, wherein the group of sensors comprises a physiological sensor and a biomechanical sensor; and wherein the processor is arranged to correlate between physiological information obtained from the physiological sensor and biomechanical information obtained from the biomechanical sensor to determine a validity of the potential medical event.

15. The portable monitoring unit according to claim 13, wherein the group of sensors comprises an environment condition sensor and a physiological sensor; and wherein the processor is arranged to correlate between physiological information obtained from the physiological sensor and environment condition information obtained from the environment condition sensor to determine a validity of the potential medical event.

16. The portable monitoring unit according to claim 13, wherein the group of sensors comprises an environment condition sensor, a biomechanical sensor and a physiological sensor; and wherein the processor is arranged to correlate between physiological information obtained from the physiological sensor, biomechanical information obtained from the biomechanical sensor and environment condition information obtained from the environment condition sensor to determine a validity of the potential medical event.

17. The portable monitoring unit according to claim 13, wherein the group of sensors comprises two types of physiological sensors; and wherein the processor is arranged to correlate between physiological information obtained from the two types of physiological sensors to determine a state of a monitored person.

18. The portable monitoring unit according to claim 13, further comprises a transmitter; wherein the processor is arranged to instruct the transmitter to transmit a potential event information in response to a detection of the potential medical event.

19. The portable monitoring unit according to claim 18, wherein the transmitter is arranged to attempt to transmit the potential event information and to generate a transmission failure indication of the attempt fails; wherein the processor is arranged to provide an alert that induces a suggestion to change a location of the monitored person or to inform a medical center of the failure by means that differ from the transmitter.

20. The portable monitoring unit according to claim 18, comprising a positioning module arranged to determine a position of the portable monitoring unit; wherein the transmitter is arranged to attempt to transmit the potential event information and to generate a transmission failure indication of the attempt fails; wherein the processor is arranged to determine a preferred transmission location and generate an alert to a monitored person indicative of the preferred transmission location; wherein a probability of transmission failure from the preferred transmission location is substantially lower than a probability of a transmission failure at a current location of the monitored person.

21. The portable monitoring unit according to claim 18, wherein the processor is arranged to activate the transmitter and instruct the transmitter to perform long range transmission of the potential event information in response to a detection of the potential medical event.

22. The portable monitoring unit according to claim 18, comprising a transmitter, a primary power source and a secondary power source; wherein the processor is arranged to prevent a transmission of potential event information during a low power period during which the primary power source is empty; and to initiate a transmission of potential event information obtained during the low power period.

23. The portable monitoring unit according to claim 13, wherein the processor is arranged to receive the raw medical information in a continuous manner.

24. A portable monitoring unit, comprising:
a memory module for storing a media clip that comprises instructions related to the operation of the monitoring unit, for storing raw medical information, and for storing medical event information; and
a processor arranged to receive raw medical information from at least one primary sensor and to process the raw medical information and detect potential medical events, to request additional information from at least one secondary sensor in response to a detection of the potential event, wherein the at least one primary sensor and the at least one secondary sensor form a group of sensors;
wherein the group of sensors comprises an environment condition sensor, a biomechanical sensor and a physiological sensor; and
wherein the processor is arranged to correlate between physiological information obtained from the physiological sensor, biomechanical information obtained from the biomechanical sensor and environment condition information obtained from the environment condition sensor to determine a validity of the potential medical event.

25. A portable monitoring unit, comprising:
a memory module for storing raw medical information, and for storing medical event information;
a transmitter; and
a processor arranged to receive raw medical information from at least one primary sensor and to process the raw medical information and detect potential medical events, and to instruct the transmitter to transmit a potential event information in response to a detection of the potential medical event;
wherein the transmitter is arranged to attempt to transmit the potential event information and to generate a transmission failure indication if the attempt fails; and
wherein the processor is arranged to provide an alert that induces a suggestion to change a location of the monitored person or to inform a medical center of the failure by means that differ from the transmitter.

26. The portable monitoring unit according to claim 25, comprising a positioning module arranged to determine a position of the portable monitoring unit; wherein the processor is arranged to determine a preferred transmission location and generate an alert indicative of the preferred transmission location; wherein a probability of transmission failure from the preferred transmission location is substantially lower than a probability of a transmission failure at a current location of the monitored person.

27. The portable monitoring unit according to claim 25, comprising a positioning module arranged to determine a position of the portable monitoring unit; wherein the memory module stores positions of preferred transmission locations and wherein the processor is arranged to select a preferred transmission location based on the position of the portable monitoring unit and generate an alert indicative of the preferred transmission location; wherein a probability of transmission failure from the preferred transmission location is substantially lower than a probability of a transmission failure at a current location of the monitored person.

28. A method, comprising:
storing in a memory module of a portable monitoring unit, a media clip that comprises instructions related to the operation of the monitoring unit;
displaying the media clip;
receiving, by the portable monitoring unit, raw medical information from at least one primary sensor;
processing the raw medical information to detect potential medical events; and
performing, by the processor, a potential event related operation in response to a detection of the potential medical event.

29. The method according to claim 28, comprising requesting additional information from at least one secondary sensor in response to a detection of the potential event, and processing the additional information to determine a validity of the potential medical event.

30. The method according to claim 29, comprising correlating between physiological information obtained from a physiological sensor and biomechanical information obtained from a biomechanical sensor to determine a validity of the potential medical event.

31. The method according to claim 29, comprising correlating between physiological information obtained from a physiological sensor and environment condition information obtained from an environment condition sensor to determine a validity of the potential medical event.

32. The method according to claim 29, comprising correlating between physiological information obtained from a physiological sensor, biomechanical information obtained from a biomechanical sensor and environment condition information obtained from an environment condition sensor to determine a validity of the potential medical event.

33. The method according to claim 29, comprising correlating between physiological information obtained from two types of physiological sensors to determine a state of a monitored person.

34. The method according to claim 28, comprising attempting to transmit, by a transmitter of the portable monitoring unit, a potential event information in response to a detection of the potential medical event.

35. The method according to claim 34, comprising generating a transmission failure indication if the attempting to transmit fails; and provide an alert that induces a suggestion to change a location of the monitored person or to inform a medical center of the failure by means that differ from the transmitter.

36. The method according to claim 34, comprising determining, by a positioning module, a position of the method; attempting to transmit the potential event information; generating a transmission failure indication if the attempting to transmit fails; determining a preferred transmission location; and generating an alert to a monitored person indicative of the preferred transmission location; wherein a probability of transmission failure from the preferred transmission location is substantially lower than a probability of a transmission failure at a current location of the monitored person.

37. The method according to claim 34, comprising activating the transmitter and instructing the transmitter to perform long range transmission of the potential event information in response to a detection of the potential medical event.

38. The method according to claim 28, comprising preventing a transmission of potential event information during a low power period during which the primary power source is empty; and initiating a transmission of potential event information obtained during the low power period.

39. The method according to claim 28, comprising receiving the raw medical information in a continuous manner.

40. A method, comprising: receiving, by the portable monitoring unit raw medical information from at least one primary sensor; processing the raw medical information to detect potential medical events; requesting additional information from at least one secondary sensor in response to a detection of the potential event, and processing the additional information to determine a validity of the potential medical event.

41. The method according to claim 40, comprising requesting additional information from at least one secondary sensor in response to a detection of the potential event, and processing the additional information to determine a validity of the potential medical event.

42. The method according to claim 41, comprising correlating between physiological information obtained from a physiological sensor and biomechanical information obtained from a biomechanical sensor to determine a validity of the potential medical event.

43. The method according to claim 41, comprising correlating between physiological information obtained from a physiological sensor and environment condition information obtained from an environment condition sensor to determine a validity of the potential medical event.

44. The method according to claim 41, comprising correlating between physiological information obtained from a physiological sensor, biomechanical information obtained from a biomechanical sensor and environment condition information obtained from an environment condition sensor to determine a validity of the potential medical event.

45. The method according to claim 41, comprising correlating between physiological information obtained from two types of physiological sensors to determine a state of a monitored person.

46. The method according to claim 40, comprising attempting to transmit, by a transmitter of the portable monitoring unit, a potential event information in response to a detection of the potential medical event.

47. The method according to claim 46, comprising generating a transmission failure indication if the attempting to transmit fails; and provide an alert that induces a suggestion to change a location of the monitored person or to inform a medical center of the failure by means that differ from the transmitter.

48. The method according to claim 46, comprising determining, by a positioning module, a position of the method; attempting to transmit the potential event information; generating a transmission failure indication if the attempting to transmit fails; determining a preferred transmission location; and generating an alert to a monitored person indicative of the preferred transmission location; wherein a probability of transmission failure from the preferred transmission location is substantially lower than a probability of a transmission failure at a current location of the monitored person.

49. The method according to claim 46, comprising activating the transmitter and instructing the transmitter to perform long range transmission of the potential event information in response to a detection of the potential medical event.

50. The method according to claim 46, comprising preventing a transmission of potential event information during a low power period during which the primary power source is empty; and initiating a transmission of potential event information obtained during the low power period.

51. The method according to claim 40, comprising receiving the raw medical information in a continuous manner.

52. A method, comprising:
receiving, by a portable monitoring unit, raw medical information from at least one primary sensor;
processing the raw medical information to detect potential medical events;
requesting additional information from at least one secondary sensor in response to a detection of the potential event; wherein the at least one primary sensor and the at least one secondary sensor form a group of sensors; and
correlating between physiological information obtained from a physiological sensor of the group of sensors, biomechanical information obtained from a biomechanical sensor of the group of sensors, and environment condition information obtained from an environment condition sensor of the group of sensors to determine a validity of the potential medical event.

53. A method, comprising:
receiving, by the portable monitoring unit, raw medical information from at least one primary sensor;
processing the raw medical information to detect potential medical events;
requesting additional information from at least one secondary sensor in response to a detection of the potential event;
processing the additional information to determine a validity of the potential medical event; and
attempting to transmit, by a transmitter of the portable monitoring unit, a potential event information in response to a detection of the potential medical event.

54. The method according to claim 53, comprising generating a transmission failure indication if the attempting to transmit fails; and provide an alert that induces a suggestion to change a location of the monitored person or to inform a medical center of the failure by means that differ from the transmitter.

55. The method according to claim 54, comprising determining, by a positioning module, a position of the method; attempting to transmit the potential event information; generating a transmission failure indication if the attempting to transmit fails; determining a preferred transmission location; and generating an alert to a monitored person indicative of the preferred transmission location; wherein a probability of transmission failure from the preferred transmission location is substantially lower than a probability of a transmission failure at a current location of the monitored person.

56. The portable monitoring unit according to claim 7, wherein the transmitter is arranged to transmit an indication about a completion of a measurement by the portable monitoring unit to a third party.

57. The portable monitoring unit according to claim 7, wherein the transmitter is arranged to transmit an indication about an incompletion of a measurement by the portable monitoring unit to a third party.
